(19) United States
(12) Patent Application Publication
(10) Pub. No.: US 2016/0287646 A1
(43) Pub. Date: Oct. 6, 2016

(54) MACROALGAE COMPOSITIONS, PROCESSES FOR PREPARATION THEREOF, AND USES IN SPORTS NUTRITION

(71) Applicant: OmniActive Health Technologies (Canada) Limited, Charlottetown (CA)

(72) Inventors: Jayant DESHPANDE, Charlottetown (CA); Khadija GHANAM, Charlottetown (CA); Vandita SRIVASTAVA, Pune (IN)

(21) Appl. No.: 15/087,212

(22) Filed: Mar. 31, 2016

(30) Foreign Application Priority Data
Mar. 31, 2015 (IN) 1313/MUM/2015

(51) Int. Cl. A61K 36/03; A61K 31/20; A61K 31/202; A61K 36/04; A61K 31/201

(52) U.S. Cl. CPC A61K 36/03; A61K 36/04; A61K 31/201; A61K 31/202; A61K 31/20; A61K 2236/39

(57) ABSTRACT

Macroalgae compositions, processes for the preparation thereof, and applications thereof as a sports nutrition supplement are described. More particularly, a macroalgae composition includes a macroalgae extract including medium chain and long chain fatty acids in a ratio of about 1:0.0005 to 1:0.1. The composition is prepared by extracting macroalgae selected from green, red and/or brown algae or the combinationsusing suitable non-polar, semi-polar and polar organic and/or inorganic solvents and the combinations thereof. The composition is useful as a sports nutrition supplement in an effective daily dose, and enhances exercise endurance, resistance to muscle fatigue and/or supports protein synthesis performance. The composition is safe for human consumption and is useful as sports nutrition and for body building applications.

### FIELD

Macroalgae compositions, processes for the preparation thereof and applications thereof in sports nutrition are described herein. More particularly, Macroalgae compositions herein include a macroalgae extract, comprising medium and long chain fatty acids in a ratio, for example an area percentage ratio, of about 1:0.0005 to 1:0.1. The fatty acids may be selected from the group of, but not limited to lauric acid, myristic acid, palmitic acid, arachidic acid, linoleic acid, stearic acid, oleic acid, linolenic acid, eicasanoic acid and the like. Compositions herein are prepared by extracting macroalgae selected from green, red and/or brown seaweeds or the combinationsusing suitable non-polar, semi-polar and polar organic and/or inorganic solvents and the combinations thereof. Compositions as described herein are useful in sports nutrition through the administration thereof in an effective daily dose, and enhance exercise endurance, resistance to muscle fatigue, and/or support protein synthesis performance. Macroalgae extract also exhibits effect on various biomarkers such as IGF-1 expression, cortisol, mitochondrial mass, mitochondrial oxygen consumption, PGC1-alpha production, ATP production, glucose uptake, and glycogen synthesis. Compositions herein are comprised of a specific ratio of medium chain fatty acids and long chain fatty acids, are safe for human consumption, and are prepared by economical and industrially viable processes, and are useful in sports nutrition and/or for body building applications.

### BACKGROUND

Modern hectic lifestyles and busy schedules in urban civilization are compelling current generation to opt for ways to lessen the stress and live healthy and happy life. Life has become very demanding due to high expectations from personal life and career goals, thus putting a lot of burden on people. Generally people tend to spend very less attention to their health and stress busters such as sports or some form of exercise are neglected or given less priority. Level of exertion increases to such a level that, even if at some time point people realize or are advised to initiate in more sports and/or exercise activity, people may lack adequate stamina and energy required to continue these activities. Same conditions may be experienced by athletes, who experience fatigue during their routine practice sessions or even during competition events, thus leading to failure. This highlights the need of awareness of sports nutrition.

Sports nutrition is a science that requires a good understanding of nutritional factors effecting performance, recovery and health, a knowledge of the nutritional value of food and fluids, and the necessary skills to implement appropriate nutritional strategies into daily training and competition. A good based diet provides adequate nutrients and energy to enhance adaptations from training, support optimal recovery and/or avoid excessive food-related stress. Heavy training increases the need for nutrients, particularly carbohydrate, protein and micronutrients (vitamins and minerals). Athletes are now encouraged to plan their post exercise nutrition as diligently as their preparation and performance nutrition. The importance of nutrition after exercise has expanded from strategies to recover or replace carbohydrate energy stores to include methods to enhance muscle repair and adaptation. The pressures of training and busy lifestyles can make it difficult to optimize nutrition with regular foods and there is an increased understanding how certain supplements may benefit athletic performance.

Athletes as well as common persons willing to continue normal exercise have a need for safe and effective nutritional sports supplements. Although some options are already available in the market, there are certain side-effects of the existing sport nutrition such as sports drinks that are based on mid-length carbohydrates, such as maltodextrin. These sport drinks exert a high osmotic pressure in the stomach, which can result in bloating, nausea, and vomiting, as well as further dehydrating the person by pulling water out of the blood into the stomach. Sugary sports drinks and other sports drinks that are based on mid-length carbohydrates are not designed for a steady, sustained release of energy.

Various products are available in the marketplace as supplements for bodybuilders, elite, and recreational athletes. These can be categorized as protein and non-protein products. Protein products are consumed as powders, bars, and ready-to-use-drinks primarily to support muscle protein synthesis. Milk proteins (e.g. whey and casein) have been traditionally used for this purpose but recently plant proteins are emerging as vegan alternatives. The use of both whole, intact proteins and hydrolysates are common, with the latter being more expensive but having enhanced bioavailability. Non-protein ingredients include creatine, beta-alanine, and amino acid mixtures. The non-protein ingredient segment is largely directed at augmenting muscle protein synthesis, improving endurance, and/or providing hydration. Omega-3 supplements are popular for athletes and non-athletes alike for their ability to improve endothelial (blood vessel) function, reduce inflammation, and/or increase provision of energy from fat. Fish oil, rich in omega-3 polyunsaturated fatty acids (PUFAs), exerts anti-inflammatory and immunomodulatory effects and may be useful as a nutritional countermeasure to exercise-induced inflammation and immune dysfunction in athletes. (Ref: International Journal of Sport Nutrition and Exercise Metabolism, 2013, 23, 83-96). However this source of fatty acids may not be acceptable to all because of the typical smell or odor, and thus can affect their palatability.

There is a significant opportunity for natural ingredients that augment muscle protein synthesis leading to increased muscular build. Body builders consume large amounts of protein per day, with a low efficiency of incorporation or absorption. Protein hydrolysates that provide a more readily digestible protein source may prove better for this purpose. However use of hydrolysates increase cost of product and the product becomes unpalatable due to use of peptide components.

An alternative approach is the use of a protein synthesis stimulator that encourages dietary protein incorporation into muscle protein. An emerging trend in the industry is the use of products that stimulatem TOR (mammalian Target of Rapamycin), a cellular signaling molecule that has been implicated in the regulation of protein synthesis. For example, the branched-chain amino acids (leucine, isoleucine and valine), in addition to their role as substrates in protein synthesis, also stimulate mTOR and are marketed for that specific purpose. But palatability still remains the issue in this approach. Thus there is need of alternate, effective, and palatable sports nutrition from a natural source, which can provide sustainable endurance and avoid muscle soreness to persons for normal daily exercise activities as well as to athletes.

Macroalgae or seaweeds are the products which are known for their high nutritional values and they are rich in fibers, vitamins and proteins. It is also known as a carbohydrate rich diet and is used in soups, salads and in confectionary items such as jellies and puddings. It is easily available along the sea shores and is known for many health applications. There are various references which describe processes for the extraction of macroalgae or seaweeds and their various applications.

WO2011057406A1 relates to protein concentrates and protein isolates comprising combinations of proteins, peptides and amino acids, as well as processes for their production. In particular, the reference relates to a process for removing fiber from macroalgae and/or microalgae to produce edible protein products.

US20120189706 relates to products derived from the exudate of kelp as well as high-purity fucoidan derived from harvested kelp, specifically the brown algae Macrocystis pyrifera. More particularly, the reference relates to brown algae exudate and Macrocystis pyrifera derived pharmaceutical, nutraceutical, and cosmeceutical products and additives for oral, topical, and transdermal delivery to treat, prevent, or aid the health and wellness of an individual as well as the use of brown algae exudate and Macrocystis pyrifera high-purity fucoidan as antioxidant additives for use with or in food, beverage, desert, or cosmetic products or in combination with other nutraceutical and cosmeceutical products to boost their final antioxidant impact.

WO2012089615A1 relates to a method of processing macroalgae matter to provide a nutraceutical preparation, the process comprises: a) heating a body of liquid containing the macroalgae matter during a first time period, b) collecting leached liquid from the macroalgae matter during the first time period, c) applying a hot liquid extraction step to the macroalgae matter during a second time period resulting in a liquid extract, the second time period occurring after the first time period has elapsed, and d) combining the leached liquid with the liquid extract.

US20080280994A1 relates to hot water extract of Ascophyllum, comprising at least about 20% by weight of polyphenolic compounds. Prior to aqueous extraction, lipophilic components are removed from the Ascophyllum by using hexane solvent and the remaining part is extracted with hot water. The aqueous extract is useful for inhibiting alpha-glucosidase activity; preventing or treating conditions mediated by alpha-glucosidase activity; reducing blood glucose levels; preventing or treating diabetes; modulating glucose uptake in adipocytes; preventing or treating obesity; scavenging free radicals; stimulating the immune system; activating macrophages; preventing or treating condition mediated by macrophage activation; and modulating nitric oxide production by macrophages.

WO2014083141A1 relates to an aqueous extraction process of active ingredients from the algae Ascophyllum nodosum for obtaining a stable extract of Ascophyllum nodosum. Further the stable aqueous extract may comprise ethanol up to a maximum of 20% in volume, and sulphated polysaccharides with high molecular weight.

US20150190950A1 relates to a method for preparing a powder from brown macroalgae, comprises treating the brown macroalgae with weak acids, followed by filtration, grinding and then drying the residue so as to obtain a powder with a particle size of between 0.5 and 1.5 mm wherein said macroalgae is chosen from among brown algae from the laminariales or fucales order.

US20130266522A1 relates to a composition for oro-dental use based on an aqueous extract of brown algae comprising a mixture of algae with at least Ascophyllum nodosum and Fucus species combined with at least one silver-loaded zeolite.

WO2014078300A1 relates to a cosmetic active ingredient for improving the appearance of the skin beneath the eyes comprising an aqueous extract of Fucus vesiculosus.

US20070036821A1 relates to a method for increasing anti-thrombotic activity in a mammal comprising the step of administering to a mammal an amount of seaweed extract, prepared using water as solvent, in effective doses; wherein the seaweed is selected from the group consisting of Fucus vesiculosus, Fucus Evanescens, Laminaria brasiliensis, or Ascophylum nodosum.

US20050196410A1 relates to a method for retardation of inflammation in a mammal comprising the step of administering to a mammal an amount of aqueous seaweed extract in effective doses; wherein the seaweed is selected from the group consisting of Fucus vesiculosus, Fucus evanescens, Laminaria brasiliensis, or Ascophylum nodosum.

### SUMMARY

The references above relate to uses of macroalgae compositions, which are prepared by employing solvents such as water and its application as biofuels, for the treatment of diabetes, obesity, and skin infections. However, the references do not relate to uses of macroalgae extracts or compositions for enhancement of exercise endurance or body building applications as in sports nutrition.

Macroalgae or seaweeds are valuable food sources because of their high nutritional value when compared to land plants. Seaweeds are being utilised as a raw material in functional foods. It is known to extract bioactive constituents from seaweed, which are then used in the preparation of functional foods. However, the extraction methods known heretofore typically require a long extraction time, use solvents that are generally toxic and inflammable, and need a very high temperature and/or pressure.

As reported herein exhaustive trials have been carried out for the preparation of compositions from particular macroalgae, employing various solvents such as polar, semi-polar and/or non-polar solvents, and the combinations thereof. The processes, described herein are convenient and economic as the extraction is carried out in minimum time, does not use high temperature or pressure, and employs safe solvents for extraction, so that the composition is safe for human consumption. The composition, including an extract, is characterized for its chemical constituents and evaluated for its activity in sports nutrition. The compositions herein are comprised of medium chain fatty acids and long chain fatty acids in a specific ratio, for example an area percentage ratio, of at or about 1:0.0005 to at or about 1:0.1, thus making it suitable and effective for use as an energy store to improve performance of athletes.

Thus the processes and compositions herein are economical, making use of commonly available commercial equipment for their preparation, and provide safe alternative sources of natural supplements for sport nutrition.

In an embodiment, a macroalgae composition is provided, which when administered to a subject in an effective amount, is useful for sports nutrition.

In an embodiment, a macroalgae composition is provided and comprises an extract, wherein the macroalgaeis selected from the group of, but not limited to various genera of green, red, brown seaweeds.

In an embodiment, the composition is comprised of medium chain fatty acids and long chain fatty acids in a ratio, for example a ratio, for example an area percentage ratio, of at or about 1:0.0005 to at or about 1:0.1.

In an embodiment, the composition includes macroalgae extract, which is comprised of fatty acids selected from the group of, but not limited to lauric acid, myristic acid, palmitic acid, arachidic acid, linoleic acid, stearic acid, oleic acid, linolenic acid and the like or the combination thereof.

In an embodiment, the macroalgae is preferably selected from various genera of brown seaweeds such as Ascophyllum, Fucus, Furcelleria, Laminaria and the like or combinations thereof. More particularly, the composition may be comprised of the macroalgae extract obtained from species of brown seaweeds such as Ascophyllum nodosum, Fucus vesiculosus and other species of Furcelleria and Laminaria.

In an embodiment, an industrially viable process for preparation of macroalgae composition is provided by employing non-polar, semi-polar, polar solvents or combinations thereof in suitable volumes.

In an embodiment, a macroalgae composition is provided by being prepared using non-polar, semi-polar and/or polar solvents or the combinations thereof, which is comprised of chemical constituents of varying polarity.

In an embodiment, macroalgae compositions herein comprise chemical constituents, which are characterized by various analytical methods.

In an embodiment, macroalgae compositions are provided and their effect(s) evaluated relative to health parameters related to sports nutrition.

In an embodiment, macroalgae compositions herein are effective in sports nutrition in a daily dose and enhance exercise endurance, resistance to muscle fatigue and/or support protein synthesis performance. Macroalgae extract also exhibits effect on various biomarkers such as insulin-like growth factor 1 (IGF-1) expression, cortisol, mitochondrial mass, mitochondrial oxygen consumption, peroxisome proliferator-activated receptor gamma coactivator 1-alpha (PGC1-alpha) production, adenosine triphosphate (ATP) production, glucose uptake and/or glycogen synthesis.

In an embodiment, macroalgae compositions herein enhance exercise endurance by enhancing mitochondrial biogenesis and also enhance biomarkers responsible for protein synthesis.

In an embodiment, an industrially viable process for the preparation of the composition comprised of extract is provided, wherein organic and/or organic solvents of varying polarity are used for the extraction. The extract is further comprised of biologically active chemical constituents such as medium chain and long chain fatty acids. The composition is evaluated for its effect on health parameters related to sports nutrition.

Macroalgae composition as described herein includes macroalgae extract, which is comprised of medium chain and long chain fatty acids in a ratio, for example an area percentage ratio, of at or about 1:0.0005 to at or about 1:0.1 and is useful for enhancing exercise endurance, resistance to muscle fatigue and protein synthesis, thus acts as a sports nutrition and bodybuilding supplement, when used in an effective amount. The composition, which is essentially macroalgae extract is prepared by solvent extraction and then evaluated for its effect on various biomarkers related to sports nutrition. Macroalgae composition is comprised of fatty acids selected from the group of, but not limited to lauric acid, myristic acid, palmitic acid, behenic acid, arachidic acid, linoleic acid, stearic acid, oleic acid, linolenic acid and the like or the combination thereof.

More particularly a macroalgae composition herein is comprised of medium chain fatty acids and long chain fatty acids in a ratio, for example an area percentage ratio, of at or about 1:0.001 to at or about 1:0.01.

Medium chain fatty acids are selected from the group of, but not limited to Caproic acid (C-6), Caprylic acid (C-8), Capric acid (C-10), Lauric acid (C-12) and combinations thereof.

Long chain fatty are selected from the group of, but not limited to Myristic Acid (C-14), Palmitic Acid (C-16), Heptadecanoic Acid (C-17), Stearic Acid (C-18), Arachidic Acid (C-20), Palmitoleic Acid (C-16:1), Oleic Acid (C-18:1), Linoleic Acid (C-18:2), Linolenic Acid (C-18:3), Eicasanoic Acid (C-20:1) and combinations thereof.

In an embodiment, a process for preparation of macroalgae compositions provided, which is convenient, economic, does not use high temperature or pressure, and employs safe solvents for extraction, so that the resulting composition is safe for human consumption. The composition, which includes an extract, is characterized for its chemical constituents and evaluated for its activity as a sports nutrition supplement. Thus economically viable processes are described herein, which make use of available commercial equipment and provides safe alternative source of natural supplement for sport nutrition.

The extract is prepared by treating green, brown or red seaweeds with suitable non-polar, semi-polar and polar organic and/or inorganic solvents and the combinations thereof. The composition is comprised of medium chain and long chain fatty acids in a ratio, for example an area percentage ratio, of at or about 1:0.0005 to at or about 1:0.1 and it is useful as sports nutrition supplement in an effective daily dose, and enhances exercise endurance, resistance to muscle fatigue and/or supports protein synthesis performance.

The effective daily dose for administration ranges from at or about 250 mg to at or about 4000 mg of the medium and chain and long chain fatty acids in the defined ratio, and it can be administered to the subjects who are in need of such supplement or the subjects carrying out daily routine activities or the sports activities such as running, cycling, swimming or any other sports activities which need endurance and/or stamina. In an embodiment, the daily does can range from at or about 500 mg to at or about 3000 mg.

In an embodiment, a period of daily doses can range for example but not limited to at or about 3 months to at or about 2 years. It will be appreciated that there may be no fixed time period for the daily doses as it may be less or longer than such range.

Macroalgae extract, when administered in an effective daily dose, exhibits effect on various biomarkers such as IGF-1 expression, cortisol, mitochondrial mass, mitochondrial oxygen consumption, PGC1-alpha production, ATP production, glucose uptake and/or glycogen synthesis. This results in enhanced mitochondrial biogenesis through increased mitochondrial mass and mitochondrial oxygen consumption. The composition is safe for human consumption, prepared by economical and industrially viable process and it is useful as sports nutrition and for body building applications.

### DETAILED DESCRIPTION

Described herein are macroalgae compositions, processes for their preparation, and of their evaluation as a sports nutrition supplement.

As used herein, the term “Macroalgae composition” refers to a product which includes active material obtained by solvent extraction of seaweeds preferably brown, green or red marine algae or seaweeds; the composition is thus includes macroalgae extract. The brown, green or red marine algae are selected from the group of, but not limited to genera such as Ascophyllum, Fucus, Laminaria, Furcellaria, Sargassum, Chondrus, Caulerpa, Codium, Gracileria, Macrocystis, Monostroma, Porphyra, Cladophora, Halimeda, Bryopsis, Chaetomorpha and the like or the combinations thereof. Seaweed refers to several species of macroscopic, multicellular, marine algae that are found near the seabed. Macroalgae belong to mainly three classes of seaweeds such as Phaeophyta, Chlorophyta and Rhodophyta and various genus and species belonging to these three classes.

In an embodiment, macroalgae compositions herein are comprised of a macroalgae extract, which can be administered either as such or can be suspended in a suitable oil medium. The extract can be also formulated in the form of beadlets or spray dried powders and can be compressed in tablets or filled in capsules. For formulations into other types of finished dosage forms, a variety of excipients can be used such as binder, filler, disintegrant, diluents, carrier, glidant.

The fatty acids extracted from the macroalgae do not exist in the desired ratios as stated herein, but where the macroalgae is specifically extracted as per the preparation methods herein, followed by its derivatization as ester (esterification) and quantification by gas chromatography so as to obtain the ratio of fatty acids and evaluate them. This enables quantification in the form of medium chain and long chain fatty acids. Residual solvents or other components remaining after the extraction, if any, do not impact the effectiveness of the extract for the composition.

The composition which includes a macroalgae extract is prepared by employing food grade solvents, which are safe for human consumption. The solvents may be non-aqueous or sometimes the combination of solvents with water can be used for the extraction.

Macroalgae compositions herein are comprised of biologically active medium chain and long chain fatty acids in a ratio, for example an area percentage ratio, of at or about 1:0.0005 to at or about 1:0.1, wherein the fatty acid are selected from the group of, but not limited to lauric acid, myristic acid, palmitic acid, arachidic acid, linoleic acid, stearic acid, oleic acid, linolenic acid, eicasanoic acid and the like or the combination thereof. Medium chain and long chain fatty acids may be saturated or unsaturated fatty acids. The quantification of these fatty acids is carried out in terms of area percentage by using chromatographic technique.

The term ‘medium chain fatty acid” used herein refers to fatty acids having 6-12 carbon atoms in the chain. Fatty acids found in medium chain triglycerides (MCTs) are called medium chain fatty acids.

The term ‘long chain fatty acid” used herein refers to fatty acids which have with tails of 13 to 21 carbons.

Long chain fatty acids can act as depot of energy in body which is useful for sports nutrition application, as they provide instant energy to athletes, sportsmen in order to enhance endurance performance and avoid muscle fatigue.

Medium chain fatty acids are selected from the group of, but not limited to Caproic acid (C-6), Caprylic acid (C-8), Capric acid (C-10), Lauric acid (C-12) and combinations thereof.

Long chain fatty are selected from the group of, but not limited to Myristic Acid (C-14), Palmitic Acid (C-16), Heptadecanoic Acid (C-17), Stearic Acid (C-18), Arachidic Acid (C-20), Palmitoleic Acid (C-16:1), Oleic Acid (C-18:1), Linoleic Acid (C-18:2), Linolenic Acid (C-18:3), Eicasanoic Acid (C-20:1) and combinations thereof.

The term “sports nutrition” as used herein refers to the compositions or nutritional supplements which are employed for supporting or enhancing performance in sports activities by increasing physical fitness, when administered in an effective daily amount. The desired physical fitness or support for carrying out strenuous physical activities or sports activities is related to enhancing stamina, enhancing exercise duration and endurance, increasing muscle resistance to fatigue, enhancing protein synthesis for body building and also increasing resistance to soreness, fatigue and muscle injury, thus enabling an athlete to perform better in their activities. Such compositions or nutritional supplements also help to increase endurance of non-sports person when they are undergoing strenuous activities in their daily routine.

Macroalgae compositions, as described herein relate to the composition which includes a macroalgae extract comprised of medium chain and long chain fatty acids in a ratio of at or about 1:0.0005 to at or about 1:0.1 and which is useful as a sports nutrition supplement. A process for preparation of macroalgae composition is provided, comprising desired chemical constituents. The composition is prepared by treating suitable seaweed(s) with suitable solvents at specific conditions to form an extract which is rich in fatty acids in the specific ranges.

Macroalgae as described herein are marine algae and/or seaweeds, which are classified into three broad groups or classes, based on pigmentation and other characteristics, such as green Chlorophyceae, (2) Brown—Phaeophyceae, and (3) Red—Rhodophyceae. Macrolagae compositions herein include an extract, obtained from brown, red and green seaweed varieties. Macroalgae compositions as described herein, are preferably prepared by extraction of brown macroalgae selected from the group of, but not limited to, Ascophyllum, Laminaria, Fucus, Furcellaria, Undaria, Himanthalia, Alaria, Saccharina; preferably the brown seaweed species are selected from Furcellaria lumbricalis, Laminaria japonica, Fucus vesiculosus, Ascophyllum nodosum, Laminaria saccharina, Laminaria digitata, Himanthalia elongate, Undaria pinnatifida, Macrocystis pyrifera, Alaria fistulosa, Alaria marginata, Fucus evanescens, Laminaria cichorioides, Laminaria digitata, Laminaria hyperboria, Saccharina latissima and the like. Macroalgae are harvested, for example from different Atlantic Canada sources or Norwegian Sea beaches or any other suitable sea shores and are used for the preparation of macroalgae compositions herein.

Macroalgae has been used in European countries as a rich source of carbohydrates, because it contains carragenans, alginates and arabinose. It is also known as an alkaline food and therefore helps in maintaining acid base balance in the body and an effective component in a healthy acid-alkaline diet. It also contains high natural iodine levels and has been used to treat hypothyroidism. It is also useful for the treatment of cancer such as breast, endometrial and ovarian cancers. In addition, macroalgae is known to exert the properties such as antioxidant, anti-inflammatory, anti-tumor, antibiotic, antiviral, and it is known for applications in weight management, cholesterol reduction and cardiovascular complications.

In an embodiment, a composition herein may be obtained by carrying out extraction using organic and/or inorganic solvents and the combinations thereof, which are safe for human consumption. The process for preparation of macroalgae composition is comprised of mixing the powdered plant material with a suitable amount of solvent with or without stirring at ambient or elevated temperature, ranging from at or about 25 to at or about 70° C.

In an embodiment, a ratio of raw material to solvent may range from at or about 1:2 to at or about 1:20. In an embodiment, the ratio may be at or about 1:5 to at or about 1:15.

For example the ratio is of powdered macroalgae raw material mixed with 6-10 volumes of solvent and stirred well. The system is subjected to filtration using conventional filtration assembly. Filtrate containing extract is separated and the retained material is again mixed with definite volumes of solvent for re-extraction.

In an embodiment, the system of the raw material mixed with solvent may be subjected to filtration and the filtrate is separated and kept aside. In an embodiment, the residue or the material retained on the filter is again subjected to fresh and/or recycled solvent treatment two or more times under similar conditions and the resulting mixture is subjected to filtration. In an embodiment, filtrates obtained from such various extraction processes are combined and concentrated to obtain the active material of the composition.

In an embodiment, the solvents used in the preparation may be selected from the group of, but not limited to non-polar, semi-polar and/or, polar solvents and the combination thereof. Accordingly, the solvents employed in the process may be selected from the group such as, but not limited to, acetone, hexane, petroleum ether (low boiling), petroleum ether (high boiling), ethyl acetate, isopropyl alcohol, ethanol, dichloromethane, methanol, acetonitrile, water and a mixture thereof, more preferably from acetone, ethanol, hexane, water, either alone or in combination thereof.

In an embodiment, the total yield of suitable organic and/or inorganic extract may ranges from at or about 0.1 to at or about 2.0%. The total yield relates to a yield of extract obtained after the processes, e.g. processes of Examples 1 to 5 discussed below.

In an embodiment, a process for preparation of macroalgae composition, which is convenient, economic, does not use high temperature or pressure and employs safe solvents for extraction, so that the resulting composition is safe for human consumption. In an embodiment, the process is carried out using commonly available commercial equipment, and provides a safe alternative source of natural supplement for sport nutrition.

In an embodiment, macroalgae compositions herein are characterized for chemical constituents such as fatty acids, such as for example by a chromatography technique in area percentage and are evaluated for health applications, using cell lines and bioassays, in order to observe the effect(s) on biomarkers related to the application. The fatty acids present in the composition are analyzed quantitatively and a ratio, for example an area percentage ratio, of medium chain fatty acids to long chain fatty acids is calculated, to define the relative amount of active material in the composition.

In an embodiment, a ratio of medium chain to long chain fatty acids in macroalgae extract is at or about 1:0.0005 to at or about 1:0.1. In an embodiment, the composition is comprised of fatty acids in such a way that the ratio of medium chain fatty acids to long chain fatty acids is at or about 1:0.001 to at or about 1:0.01.

In an embodiment, macroalgae compositions herein are evaluated for their effect on mitochondrial biogenesis through evaluation of biomarkers, such as mitochondrial oxygen consumption, mitochondrial mass, PGc1alpha, and ATP production.

Effect(s) of macroalgae composition is also evaluated in other biomarkers IGF-1, cortisol, glucose uptake and glycogen, which are related to muscle fatigue and muscle regeneration.

Mitochondria represent the principal energy source in cells, converting nutrients to energy via cellular respiration. The function and content of mitochondria increases with physical training and decreases with physical inactivity. An alteration in the rate of oxygen consumption can serve as a useful indicator of mitochondrial dysfunction. By measuring oxygen consumption, a direct and specific assessment of the functioning of the electron transport chain (the key element of oxidative phosphorylation and cellular metabolism) may be obtained. Mitochondrial abundance (mass) can also be used as an indicator of mitochondrial biogenesis.

The endocrine system has been shown to respond to changes in exercise intensity. IGF-1 is an anabolic hormone synthetized by the liver to promote protein synthesis. Over-expression of IGF-1 was shown to promote myofiber regeneration and hypertrophy. Thus IGF-1 was considered as another good molecular biomarker to assess the effect of the extracts on muscle cells metabolism.

On the other hand, as a direct result of aerobic exercise, elevated oxygen consumption contributes to an increase in glucocorticoid circulation such as cortisol. During exercise training, cortisol causes rapid mobilization of fat, protein, and carbohydrates, providing the body with resources to manage an imbalance in homeostasis. However, cortisol is catabolic, decreasing muscle growth hormones, such as IGF-1. Persistent elevation of cortisol is inhibitory to muscle glycogen re-synthesis. Overtraining can lead to chronic elevated levels of circulating cortisol resulting in muscle tissue breakdown, compromised immunity, and an increased risk of sport-related injury. In current screening process, cortisol is considered as another biomarker and assessed the effects of the extracts on cortisol production.

In an embodiment, macroalgae compositions herein are evaluated through cell based assays using mitochondrial biogenesis assays, and the expression of some target proteins and hormones. The effects are compared with positive controls.

In this study, the effects were evaluated of macroalgae extracts on mitochondrial oxygen consumption, mitochondrial mass, insulin-like growth factor-1 and cortisol production.

In an embodiment, sports endurance activity of macroalgae composition is further evaluated through protein based assays wherein mTOR (mammalian target of rapamycin) a key regulator of protein synthesis is activated by protein kinase B or Akt P70S6 kinase (p70S6k), which is responsible for the initiation of protein translation. As such, p70S6k is often used as a proxy measure of muscle protein synthesis.

In an embodiment, macroalgae compositions herein are also evaluated through cell based bioassays to check the effect on biomarkers such as ATP, peroxisome proliferator-activated receptor gamma 1-alpha (PGC-1α), glucose and glycogen synthesis, which relate to evaluation of resistance to muscle fatigue by administering these compositions.

Adenosine triphosphate (ATP) represents the energy currency in all living systems. PGC-1α is a co-activator of nuclear receptors and controls several aspects of energy metabolism. More specifically, PGC-1α is a known regulator of mitochondrial gene expression, including mitochondrial biogenesis in skeletal muscle. Extracellular glucose and intracellular glycogen are the two sources of glucose molecules available to the working muscle during exercise. The increased need for metabolic fuel in the exercising muscle is met partially through an increase in the uptake and utilization of glucose. As exercise intensity increases, breakdown of glycogen stores becomes the predominant source of glucose. In the recovery period, exercised muscles are shown to be increasingly sensitive to glucose uptake, in order to rebuild their glycogen stores.

Macroalgae compositions herein are include an extract prepared from one or more varieties of seaweeds, by employing economical, industrially viable processes and solvents safe for human consumption, thus resulting into the composition which is useful for applications as sports nutrition.

In an embodiment, compositions herein are administered to a subject in an effective amount, ranging from at or about 250 mg to at or about 4000 mg daily dose, as such in the form of extract alone or along with at least one food grade excipient. It may be also administered in the dosage form such as powder, granules, beadlets, tablets, capsules, suspensions, solutions for oral consumption or any suitable non-oral dosage forms such as topical patches.

In an embodiment, a period of daily doses can range for example but not limited to at or about 3 months to at or about 2 years. It will be appreciated that there may be no fixed time period for the daily doses as it may be less or longer than such range.

The examples given below are provided to illustrate macroalgae composition, process for preparation and application as sports nutrition. While the compositions and methods have been described in terms of illustrative embodiments, certain modifications and equivalents will be apparent to those skilled in the art and are intended to be included within the scope of the compositions and methods herein. The details and advantages of which are explained hereunder in greater detail in relation to non-limiting exemplary illustrations.

### EXAMPLES

### A. Macroalgae Composition Preparation Using Solvent Extraction Process

The examples provide an extraction process for different species of macroalgae, using suitable non-polar, semi-polar and/or polar solvents to obtain macroalgae extract composition.

### Example 1

### Preparation of Macroalgae Composition from Genus Fucus

10 kg of powdered material was weighed and taken in a reactor and about 6-10 volumes of hexane was added and stirred for 3 hours at 60-65° C. The term volumes means if x portion of raw material is used, then 6× to 10× (6 to 10 times) of solvent is used for extraction, thus here for 10 kg material, 60 liter to 100 liter of solvents will be added. The extract was filtered under vacuum through suitable filtration system. The material was re-extracted two more times using similar extraction conditions as above and the extract was filtered under vacuum. The filtrates were combined and concentrated in a reactor and distillation was carried out at 50° C. The concentrated extract was evaporated to dryness using a rotary evaporator. This was subjected to further fractionation and purification by known methods. Total yield of the extract is 0.77%.

### Example 2

### Preparation of Macroalgae Composition from Fucus

The powdered material was weighed and taken in a reactor and 10 volumes of acetone was added and stirred for 3 hours at 45-50° C. The acetone extract was filtered under vacuum using suitable filtration system. The process of extraction of the above treated material was repeated two more times using the same parameters as above and the filtrate was collected. The filtrates were combined and concentrated in a reactor and distillation was carried out at 40° C. The concentrated extract was evaporated to dryness using a rotary evaporator. This was subjected to further fractionation and purification by known methods. Total yield of acetone extract is 0.98%.

### Example 3

### Preparation of Macroalage Composition from Genus Ascophyllum

The powdered material was weighed and taken in a reactor and 10 volumes of hexane was added and stirred for 3 hours at 60-65° C. The hexane extract was filtered under vacuum employing suitable filtration system. The material retained on the filter cloth is re-extracted two more times using similar conditions and filtered to get the filtrate. The filtrates obtained from all these three extraction processes were combined and concentrated in a reactor and distillation was carried out at 50° C. The concentrated extract was evaporated to dryness using a rotary evaporator. This was subjected to further fractionation and purification by known methods. Total yield of hexane extract is about 1.74%.

### Example 4

### Preparation of Macroalgae Composition from Furcelleria

The powdered material of Furcelleria genus was weighed and taken in a reactor and 10 volumes of hexane was added and stirred for 3 hours at 60-65° C. The hexane extract was filtered under vacuum through appropriate filtration system. The material retained after filtration was re-extracted two more times using similar conditions and filtered to collect the extract. The filtrates were combined and concentrated in a reactor and distillation was carried out at 50° C. The concentrated extract was evaporated to dryness using a rotary evaporator. This was subjected to further fractionation and purification by known methods. Total yield of hexane extract is about 0.25% to 0.75%.

### Example 5

### Preparation of Macroalge Composition from Genus Laminaria

Powdered material of genus Laminaria was weighed and taken in a reactor and 10 volumes of hexane was added and stirred at 60-65° C. The hexane extract was filtered under vacuum through suitable filtration system. The retained material was re-extracted two more times and subjected to filtration using same conditions as mentioned for first time extraction. The filtrates were combined and concentrated in a reactor and distillation was carried out at 50° C. The concentrated extract was evaporated to dryness using a rotary evaporator. This was subjected to further fractionation and purification by known methods. Total yield of hexane extract is about 0.3% to 0.9%.

### Example 6

### Process for Macroalgae Composition from Genus Chondrus

Powdered material was taken in a three necked round bottom flask and about 10 volumes of hexane was added and stirred for 3 hours at 60° C. on oil bath. The hexane extract was filtered out under vacuum through appropriate filtration system. The material retained after filtration was re-extracted for two more times under similar condition and filtered out. The filtrates were combined and concentrated at 50° C. under vacuum using a rotary evaporator. This was subjected to further fractionation and purification by known methods. Total yield of hexane extract was 0.32%.

### Example 7

### Process for Macroalgae Composition from Genus Chondrus

The powdered material was taken in a three necked round bottom flask and 10 volumes of acetone was added and stirred for 3 hours at 45° C. on oil bath. The acetone extract was filtered under vacuum using a known filtration system. The material retained after filtration was re-extracted two more times and filtered out under similar conditions as mentioned above. The acetone extract was filtered under vacuum through 5 μm pore sized filter cloth on a Buchner funnel. The filtrates obtained from these 3 extractions were combined and concentrated at 45° C. under vacuum using a rotary evaporator. This was subjected to further fractionation and purification by known methods. Total yield of acetone extract was 0.52%.

The macroalgae compositions of the examples were characterized for medium and long chain fatty acid content by Gas Chromatography (GC-FID) technique. The column used was DB-FFAP (30 m×0.25 mm, 0.25 μm). The flow rate was maintained about 2.4 ml/min in Split less mode with Nitrogen as carrier gas and injection volume 1.0 μL. The attenuation was retained around −6, wherein the injector temperature and detector temperature were maintained at 220° C. and 260° C. respectively. The hold time for oven temperature was maintained 2 min for up to temperature 70° C., subsequently increased to 5 minutes for up to 240° C. with rate 5° C./min. The total run-time for analysis was maintained around 41.00 min, with air flow 400 ml/min and hydrogen flow 40 ml/min.

The standard preparation of fatty acids was first injected on gas chromatography and analyzed accordingly. Sample preparation containing about 100-200 mg of macroalgae composition was analyzed in GC using above parameters. Area percentage of medium chain and long chain fatty acids was determined by described method and the ratio was calculated. See results of Table 1.

TABLE 1
Analysis data of macroalgae composition with respect to medium chain and long chain fatty acids

| | | | Ascophylum | Chondrus | Chondrus | | Fucus |
|---|---|---|---|---|---|---|---|
| | Laminariales | Furcellaria | nodosum | crispus | crispus | Ascophylum | vesiculosus |
| | (Hexane | (Hexane | (Hexane | (Acetone | (Hexane | nodosum | (Acetone |
| Fatty acid | extract) | extract) | extract) | extract) | extract) | (Acetone | extract) |
| (% area) | Example 5 | Example 4 | Example 3 | Example 7 | Example 6 | extract) | Example 2 |
| Medium chain | | | | | | | |
| fatty acid | | | | | | | |
| Lauric acid | 2.67 | 2.4 | 0.93 | 0.08 | 0.15 | 0.92 | 0.05 |
| (c-12) | | | | | | | |
| Medium chain | 2.67 | 2.4 | 0.93 | 0.08 | 0.15 | 0.92 | 0.05 |
| fatty acids- | | | | | | | |
| total area % | | | | | | | |
| Long chain | | | | | | | |
| fatty acid | | | | | | | |
| Myristic acid | 11.00 | 7.53 | 15.63 | 14.4 | 19.36 | 15.24 | 13.77 |
| (c-14) | | | | | | | |
| Palmitic acid | 28.29 | 45.37 | 30.74 | 34.83 | 38.61 | 27.54 | 26.2 |
| (c-16) | | | | | | | |
| Heptadecanoic | 1.23 | 4.79 | 2.05 | 0.6 | 1.2 | 0.83 | 0.32 |
| acid (c-17) | | | | | | | |
| Stearic acid | 4.09 | 5.73 | 2.1 | 2.56 | 4.56 | 1.91 | 2.68 |
| (c-18) | | | | | | | |
| Arachidic acid | . . . | 0.7 | . . . | 0.12 | 1.61 | 0.04 | 0.14 |
| (c-20) | | | | | | | |
| Palmitoleic acid | 3.97 | 9.34 | 2.35 | 3.43 | 1.33 | 2.12 | 2.45 |
| (c-16:1) | | | | | | | |
| Oleic acid | 16.82 | 9.21 | 32.23 | 23.8 | 11.48 | 34.45 | 33.04 |
| (c-18:1) | | | | | | | |
| Linoleic acid | 28.01 | 3.14 | 8.68 | 5.36 | 1.57 | 8.87 | 8.73 |
| (c-18:2) | | | | | | | |
| Linolenic acid | 3.92 | 0.71 | 1.32 | 2.16 | 0.06 | 2.07 | 2.75 |
| (c-18:3) | | | | | | | |
| Eicasanoic acid | . . . | 0.95 | 0.01 | 0 | 4.5 | 1.98 | 0.55 |
| (c-20:1) | | | | | | | |
| Long chain | 97.33 | 87.47 | 95.11 | 87.26 | 84.28 | 95.05 | 90.63 |
| fatty acid-total | | | | | | | |
| area % | | | | | | | |
| Ratio of | 1:0.027 | 1:0.027 | 1:0.010 | 1:0.001 | 1:0.002 | 1:0.010 | 1:0.001 |
| medium chain | | | | | | | |
| fatty acid:long | | | | | | | |
| chain fatty acid | | | | | | | |

### B: Evaluation of Macroalgae Composition on Endurance Stamina Platform

The effects of macroalgae composition on biomarkers of sport nutrition based on endurance stamina and bodybuilding platforms were evaluated, after determination of macroalgae composition dose through cell viability measurement study.

Cell Viability Measurement:

In order to determine appropriate test input dose of macroalgae composition for endurance/stamina related assays, differentiated C2C12 myotubes were treated with a range of test input concentrations. Cell viability was assessed by MTT (3-(4,5-desethyithiazol-2-yl)-2,5-diphenyl-tetrazolium bromide) assay, a common measurement of the in vitro cytotoxicity of test inputs. Conversion of MTT reagent (yellow colour) to formazan (purple colour) by living cells provides an indication of mitochondrial activity, which is directly related to cell viability. C2C12 cells were seeded in 96-well culture plates at a density of 2×10⁴ cells/mL and induced to differentiate. After a 24-hour pre-treatment with a range of select test input concentrations, used medium was removed, replaced with MTT labeling reagent (5 mg/mL in phosphate buffered saline), and incubated for 4 hours. The purple coloured formazan crystals formed in the intact cells were then dissolved overnight with MTT solubilisation solution (10% SDS in 0.01 M HCl). After solubilisation of the formazan crystals, absorbance was measured at 570 nm with a microplate reader. Data obtained from this cytotoxicity testing allowed for dose range optimization of the test inputs (macroalgae composition) for all further assays and evaluations.

### Example 8

### In-Vitro Screens for Sports Nutrition Biomarkers in C2C12 Cells

Mouse skeletal myoblasts C2C12 cells were used as a cell model to assess mitochondrial function and IGF-1 expression. Cells were cultured as undifferentiated myoblasts in high-serum growth media DMEM (Dulbecco's Modified Eagle Medium), supplemented with 10% FBS (Fetal bovine serum) and 1% penicillin/streptomycin. Cells were grown to full confluency at 37° C. in a humidified incubator with 5% CO₂. Upon reaching full confluency, growth media was replaced with low-serum differentiation media (2% horse serum) to promote the differentiation of myoblasts into multinucleated, fused myotubes, which exhibit similar characteristics than mature muscle cells.

The effect of select test inputs was assessed using the following assays:

i) Mitochondrial Oxygen Consumption:

Extracellular oxygen consumption in differentiated C2C12 cells was measured by assessing phosphorescence of a porphyrin-based, water soluble, oxygen sensitive probe (MitoXpress®-Xtra-HS, Luxcel Biosciences). Probe fluorescence is quenched by molecular oxygen (O₂), resulting in lower probe signal. As cellular respiration reduces the concentration of O₂, probe signal increases. The rate of this increase is related to the rate of cellular oxygen consumption. C2C12 cells were seeded in 96-well culture plates at a density of 2×10⁴ cells/mL, induced to differentiate, and incubated with MitoXpress probe (1 in the presence or absence of macroalgae composition (100 ug/ml). High sensitivity mineral oil was added (100 μL/well) to increase assay sensitivity by minimizing interference from ambient O₂. Probe fluorescence was measured (excitation 380 nm, emission 645 nm) using a fluorescence plate reader.

ii) Mitochondrial Mass:

The effect of treatment with macroalgae composition on mitochondrial mass was assessed by measuring changes in fluorescent intensity in differentiated C2C12 cells. The nonylacridine orange (NAO) probe binds to cardiolipin in all mitochondria, regardless of their energetic state, providing a measure of mitochondrial mass and an indication of mitochondrial biogenesis. C2C12 cells were seeded in 96-well culture plates at a density of 2×10⁴ cells/mL, induced to differentiate, and pre-treated with or without select macroalgae composition and controls. Following treatment, media was replaced with NAO probe (100 ng/mL) and incubated for 30 mins at 37° C. in a humidified atmosphere of 5% CO₂/95% air. Probe fluorescence was measured (excitation 380 nm, emission 645 nm) using a fluorescence plate reader. Fluorescent intensity relative to untreated control was then calculated. To standardize probe fluorescence to protein content of the cells, total protein content (in μg) was assessed by bicinchoninic acid (BCA) using bovine serum albumin as standard.

iii) Insulin-Like Growth Factor-1 (IGF-1):

To evaluate the effect of treatment with macroalgae composition on insulin-like growth factor-1 (IGF-1) concentrations in differentiated C2C12 cells, the mouse IGF-1 enzyme-linked immunosorbent assay (ELISA) kit (Sigma) was utilized. The kit provides a quantitative measurement of mouse IGF-1 in cell culture supernatants by employing an antibody specific coated 96-well plate. Standards and samples were added to the coated plate and any IGF-1 present in the sample bound to the immobilized antibody. After washing away any unbound antibody, HRP-conjugated streptavidin was added to the wells. The wells were washed again, followed by addition of a colorimetric TMB reagent. Color developed in proportion to the amount of bound IGF-1. Addition of a stop solution changed the color from blue to yellow, and the color intensity was read at A₄₅₀ with a microplate reader. Blank-corrected unknown sample protein concentrations were then extrapolated from a known standard curve.

### Example 9

### In-Vitro Screens for Sports Nutrition Biomarkers in H295R Cells

H295R cells (ATCC® CRL-2128) are human adrenocortical cells, widely used as in vitro model for the study of adrenal cell physiology and metabolism. It is a human adrenal cell line that presents a steroid secretion pattern and regulation similar to primary cultures of adrenal cells.

H29R cells were seeded and grown in ATCC-formulated Dulbecco's Modified Eagle's Medium (DMEM/F-12) media supplemented with 2.5% Nu Serum, 1% ITSPre-mix (6.25 mg insulin, 6.25 mg transferrin, 6.25 mg selenium, and 5.35 mg linoleic acid) and 1% penicillin/streptomycin. Cell culture conditions were maintained at 37° C. in a humidified atmosphere of 5% CO₂/95% air. Cells were then incubated in the presence or absence of macroalgae composition for 24 h before assessing cortisol production.

The effect of select test inputs was assessed using the following assay:

i) Cortisol Production

To evaluate the effect of treatment with macroalgae composition on cortisol concentrations in H295R cells, the DetectX® (Arbor Assays) immunoassay was utilized. A cortisol standard was provided to generate a standard curve for the assay and all samples were read off the standard curve. Standards or samples were pipetted into a clear microtiter plate coated with an antibody. A cortisol-peroxidase conjugate was added to the standards and samples in the wells. The binding reaction was initiated by the addition of a monoclonal antibody to cortisol to each well. After 1 hour incubation, the plate was washed and substrate was added. The substrate reacted with the bound cortisol-peroxidase conjugate. After a short incubation, the reaction was stopped and the intensity of the generated color was detected in a microtiter plate reader capable of measuring 450 nm wavelength. The concentration of cortisol in the samples was then calculated.

### Example 10

### Effect of Macroalgae Composition on ATP Production and PGC1alpha Production, Glucose Uptake and Glycogen Synthesis Using C2C12 Cell Model Preparation of C2C12 Cell Line

C2C12 cells (ATCC® CRL-1772) were seeded in 24- or 96-well culture plates as undifferentiated myoblasts and grown to 100% confluency in ATCC-formulated Dulbecco's Modified Eagle's Medium (DMEM) supplemented with 10% heat-inactivated fetal bovine serum (FBS). Upon reaching confluency, cells were induced to differentiate from myoblasts into multinucleated, fused myotubes, which exhibit similar characteristics to mature muscle cells. Differentiation medium consisted of ATCC-formulated DMEM supplemented with 2% heat-inactivated horse serum and was changed daily during the differentiation process. Cell culture conditions were maintained at 37° C. in a humidified atmosphere of 5% CO₂/95% air. After 5 days of differentiation, cells were incubated in the presence or absence of macroalgae composition (with control). Cells were treated with macroalgae compositions and also control samples to check effect on said biomarkers. The compositions and control samples used are mentioned in the respective table, showing results of the test. The effect of select macroalgae compositions were assessed using the following assays:

Secondary assays are performed on lead extracts to further characterize their biological activities and to study their mechanism of action.

i) Adenosine Triphosphate (ATP):

To evaluate the effect of macroalgae composition on ATP levels, colorimetric assay kit (Sigma-Aldrich, St. Louis, Mo.) was used, which is an enzyme-linked approach that involves the conversion of ATP in the test sample to ADP via the action of glycerol kinase which catalyses the transfer of the phosphate group to glycerol. The reaction was monitored by use of colorimetric product whose concentration varies proportional to the amount of ATP present. Following differentiation into myotubes, C2C12 cells were lysed in ATP assay buffer and added to assigned wells in a clear 96-well flat-bottom plate. A reaction mixture containing assay buffer, ATP probe, ATP converter, and developer were added to the wells. After 30 minutes, the absorbance at 570 nm will be measured using a microplate reader, and ATP concentrations will be extrapolated from a standard curve.

ii) Peroxisome Proliferator-Activated Receptor Gamma 1-Alpha:

Concentrations of PGC-1α in differentiated C2C12 cells as a result of treatment with macroalgae composition were determined by ELISA (MyBiosource). Following overnight treatment, cell lysates were collected and centrifuged to remove cellular debris. Test samples and standards were added to a 96-well plate pre-coated with monoclonal antibody to PGC-1α. The amount of PGC-1α captured by the anti-PGC-1α antibody was determined using a biotinylated-IgG antibody/HRP-streptavidin detection system.

iii) Glucose and Glycogen:

Glucose uptake in C2C12 cells was determined by bioluminescent assay (Promega), based on the detection of 2-deoxyglucose-6-phosphate (2DG6P). When 2DG6P is added to cells, it is transported across the membrane and rapidly phosphorylated. However, further modification is not possible, resulting in accumulation within the cell. Addition of a 2DG6P detection reagent, leading to the generation of NADPH from 2DG6P, luciferin from NADPH, and light from luciferin, allows for detection of a bioluminescent signal. Overall, the luminescent signal is proportional to the concentration of 2DG6P, and thus the rate of glucose uptake. Glycogen synthesis was determined in cell culture lysates from differentiated C2C12 cells using a colorimetric assay kit from BioVision Inc. This involves the addition of glucoamylase to the samples that hydrolyzes glycogen to glucose; the glucose is then oxidized to form an intermediate that reduces OxiRed, forming a colored product with strong absorbance at 570 nm.

Results:

Mitochondrial Mass, Mitochondrial Oxygen Consumption, IGF-1 and Cortisol Expression

It was observed that macroalgae composition extracted from Fucus, Ascophyllus and Laminaria species exhibited a significant increase in mitochondrial oxygen consumption. The tested compositions were selected from and in accordance with Examples 1 to 7 and Table 1 above. This effect was comparable to caffeine and leucine, used as the positive controls. Doses were tested in vitro which were used to treat the cells.

Among these macroalgae extracts, Ascophyllum and Laminariales extracts significantly affected most of the 4 biomarkers: they increased significantly mitochondrial biogenesis and IGF-1 expression and significantly decreased cortisol synthesis (See Table-2).

TABLE 2
Lead candidates for sport nutrition-endurance stamina platform

| | | Mitochondrial | | |
|---|---|---|---|---|
| Macroalgae | Mitochondrial | Oxygen | | |
| composition | Mass | consumption | IGF-1 | Cortisol |
| Fucus vesiculosus | 137 | 146 | 149 | 76 |
| extract | | | | |
| Ascophyllum | 121 | 123 | 129.6 | 72.5 |
| nodosum extract | | | | |
| Laminariales Extract | 122 | 169 | 158 | 55 |
| Caffeine | 146 | 157 | 130 | 65 |
| Leucine | 157 | 144 | 127 | 65.3 |

### C. In Vitro Evaluation of Macroalgae Composition on Protein Synthesis Platform

The effects of macroalgae composition on biomarkers of sport nutrition based on protein synthesis platform were evaluated, wherein the compositions were tested with an initial MTT assay (MIT is a name of dye 3-(4,5-dimethylthiazol-2-yl)-2,5-diphenyltetrazolium bromide) to determine cytotoxicity and establish dose range for testing.

Test inputs chosen for the protein synthesis assay platform were assessed using BioPlex (BioRad) cell signaling assays, based on the principle of the sandwich ELISA. Capture antibodies directed against the desired biomarker were covalently coupled to magnetic beads, which reacted with the sample containing the biomarker of interest. After a series of wash steps to remove any unbound protein, a biotinylated detection antibody was added to create a “sandwich” complex. The final detection complex was formed with the addition of a streptavidin-phycoerythrin (SA-PE) conjugate, which serves as a fluorescent indicator. The relative concentration of the analyte bound to each bead is proportional to the median fluorescent intensity (MFI) of the reporter signal. Differentiated C2C12 cells were pre-treated for 2 hours with or without test inputs and controls selected for protein synthesis. Cell lysates were then collected in preparation for the assay. On day 1 of the assay, samples were incubated overnight (15-18 hours) in the presence of beads directed against the following 3 biomarkers: Akt, mTOR (the mammalian target of rapamycin), and p70S6k. On day 2, detection antibodies were added, followed by incubation with SA-PE. The beads were then re-suspended in assay buffer and read on the BioPlex MAGPIX system. The assay was repeated for specific measurement of the phosphorylated forms of the proteins of interest. The relative phosphorylation of the proteins of interest served as an indicator of the ability of the test inputs to activate protein synthesis.

Results

Macroalgae composition significantly increased the 3 biomarkers of protein synthesis viz. Phospho-PKB/PKB, Phospho-mTOR/mTOR, Phospho-p70S6k/p70S6k, their effects were more pronounced than creatine or whey protein. In Table 3 below, Phospho-PKB/PKB, Phospho-mTOR/mTOR, and Phospho-p70S6k/p70S6k are expressed as a ratio of the fluorescence of the phospho-protein divided by the fluorescence of the total protein.

TABLE 3
Effect of macroalgae composition on protein synthesis biomarkers

| Macroalgae | Phospho- | Phospho- | Phospho- |
|---|---|---|---|
| composition | PKB/PKB | mTOR/mTOR | p70S6k/p70S6k |
| Fucus vesiculosus extract | 273 | 260 | 213 |
| Furcellaria Extract | 274 | 295 | 239 |
| Laminariales Extract | 318 | 297 | 241 |
| Ascophyllum Extract | 307 | 280 | 239 |
| Laminariales Extract | 277 | 275 | 228 |
| Creatine | 191 | 192 | 162 |
| Whey protein | 200 | 187 | 172 |
| Insulin | 315 | 311 | 241 |

TABLE 4
Effect of Macroalgae composition on ATP production

| | Working Concentration | Relative |
|---|---|---|
| Composition | (ug/mL) | to Control |
| Control | N/A | 99.99 |
| Caffeine | 500 uM | 225.83 |
| Creatine | 1 uM | 233.17 |
| Fucus extract | 100 | 280.78 |
| Ascophyllum extract | 100 | 249.77 |

Relative to Control represents the effect exhibited by a macroalgae composition, number of times, as compared to control, and thus shows the effectiveness in comparison to control

TABLE 5
Effect of macroalgae composition on PGC1-alpha-production

| | Working Concentration | Relative |
|---|---|---|
| Product | (ug/mL) | to Control |
| Control | N/A | 100.01 |
| Caffeine | 500 uM | 192.96 |
| Resveratrol | 100 uM | 220.96 |
| Fucus extract | 100 | 193.57 |
| Ascophyllum extract | 100 | 181.70 |

Relative to Control represents the effect exhibited by a macroalgae composition, number of times, as compared to control, and thus shows the effectiveness in comparison to control

TABLE 6
Effect of macroalgae composition on Glucose uptake

| Glucose (G) Uptake - glucose | | Glucose Uptake - glucose + | |
|---|---|---|---|
| only | | insulin | |
| 25 mM Glucose | 100.0 | 25 mM Glucose + 1 uM | 189.5 |
| | | Insulin | |
| Glucose + Fucus extract | 112.8 | Glucose + Insulin + Fucus | 246.9 |
| | | extract | |
| Glucose + Ascophyllum | 127.3 | Glucose + Insulin + | 296.7 |
| xtract | | Ascophyllum extract | |
| Glucose + Resveratrol | 132.4 | Glucose + Insulin + RSV | 291.0 |
| (RSV) | | | |
| Glucose + Caffeine | 109.6 | Glucose + Insulin + | 198.7 |
| | | Caffeine | |
| Glucose + Leucine | 108.5 | Glucose + Insulin + | 203.7 |
| | | Leucine | |

TABLE 7
Effect of macroalgae composition on Glycogen synthesis

| Glycogen Content - glucose | | Glycogen Content - glucose + | |
|---|---|---|---|
| only | | insulin | |
| 30 mM Glucose | 100.0 | 30 mM Glucose + 100 nM | 153.6 |
| | | Insulin | |
| G + Fucus extract | 131.2 | G + I + 309 | 213.2 |
| G + Ascophyllum extract | 142.4 | G + I + 414 | 223.8 |
| G + Resveratrol (RSV) | 142.9 | G + I + RSV | 223.9 |
| G + Caffeine | 115.3 | G + I + Caffeine | 164.7 |
| G + Leucine | 111.1 | G + I + Leucine | 158.2 |

For both glucose uptake and glycogen synthesis measurement is of the percentage change compared to non-treated samples. During exercise, the increased need for metabolic fuel in muscle is met partially through an increase in the uptake and utilization of glucose. As exercise intensity increases, breakdown of glycogen stores becomes the predominant fuel source. The results above showed that fucus and ascophyllum extracts significantly enhanced glucose uptake and glycogen synthesis when the cells were incubated with glucose and insulin. The effects of these extract was similar to resveratrol (RSV), a positive control known for its effects on glucose metabolism.

### OBSERVATION

Above results confirm that macroalgae composition has significant effects on biomarkers of mitochondrial biogenesis and it modulates the mitochondrial function. Treatment of muscle cells with the composition significantly affected mitochondrial biogenesis by increasing mitochondrial mass and increasing mitochondrial oxygen consumption and enhancing PGC1-alpha production. This increase of the mitochondrial aerobic capacity was correlated with increase in oxidative capacity of the muscle by increasing ATP production, increasing in glucose uptake and in glycogen synthesis necessary for higher resistance to muscle fatigue.

We claim:

1. A macroalgae composition comprising an effective amount of macroalgae extract; the extract comprising medium chain fatty acids and long chain fatty acids in a ratio of about 1:0.0005 to about 1:0.1;
wherein the composition enhances exercise endurance, resistance to muscle fatigue and supports protein synthesis in sports nutrition.

2. The macroalgae composition of claim 1, wherein medium chain fatty acids and long chain fatty acids may be selected from lauric acid, myristic acid, palmitic acid, heptadecanoic acid, stearic acid, arachidic acid, behenic acid, palimitoleic acid, oleic acid, linoleic acid, linolenic acid, eicasanoic acid, erucic acid and combinations thereof.

3. The macroalgae composition of claim 1, wherein the effective amount exhibits increased mitochondrial biogenesis, mitochondrial mass, mitochondrial oxygen consumption, and PGC1-alpha production.

4. The macroalgae composition of claim 1, wherein the effective amount exhibits increased resistance to muscle fatigue by increasing ATP production, glucose uptake, and glycogen synthesis.

5. The macroalgae composition of claim 1, wherein the effective amount exhibits support of protein synthesis by increasing expression of insulin growth factor-1 (IGF-1), enhancing protein synthesis biomarkers, and decreasing cortisol synthesis.

6. The macroalgae composition of claim 1, wherein the effective amount is effective as a sports nutrition supplement, and that ranges from about 250 mg to about 4000 mg.

7. The macroalgae composition of claim 6, wherein the effective amount is effective as a sports nutrition supplement, and that ranges from about 500 mg to about 3000 mg.

8. The macroalgae composition of claim 1, wherein the effective amount of macroalgae extract is obtained by solvent extraction from brown, red or green macroalgae.

9. A process for preparation of macroalgae composition having an effective amount of macroalgae extract; comprising:
a) treating suitable powdered macroalgae with about 6 to 12 volumes of a solvent at a temperature condition for a time period;
b) filtering out extracted material to separate an extract obtained from step a);
c) re-treating the filtered material from step b), with suitable solvent several times and filtering out the extract;
d) combining the extracts obtained from step c), and concentrating at suitable conditions to obtain the effective amount of macroalgae extract.

10. A process for preparation of macroalgae composition having an effective amount of macroalgae extract, comprising:
a) treating red, brown or green macroalgae with about 6 to 12 volumes of polar, semipolar, or non-polar solvent for 3 hours at 60° C. on an oil bath;
b) filtering out an extract obtained from step a) under vacuum using a suitable filtration system;
c) re-treating the filtered extract from step b) with polar, semi-polar or non-polar solvent at least two more times for 3 hours at 60° C. on an oil bath;
d) combining filtrates obtained from step c), and concentrating at 50° C. under vacuum using a rotary evaporator to obtain an effective amount of the macroalgae extract.

11. The process of claim 9, wherein the solvent for extraction is selected from the group of petroleum ether (low boiling), petroleum ether (high boiling), hexane, ethanol, methanol, isopropyl alcohol, n-butanol, acetone, acetonitrile and the mixtures thereof.

12. The process of claim 9, wherein the macroalgae is selected from species of Ascophyllum, Fucus, Laminaria, Furcellaria, Sargassum, Chondrus, Caulerpa, Codium, Gracileria, Macrocystis, Monostroma, Porphyra, Cladophora, Halimeda, Bryopsis, Chaetomorpha and the combinations thereof.

13. The macroalgae composition of claim 1, wherein the extract comprising medium chain and long chain fatty acids is in a ratio of about 1:0.001 to 1:0.1.
